(19) United States
(12) Patent Application Publication
(10) Pub. No.: US 2023/0016731 A1
(43) Pub. Date: Jan. 19, 2023

(54) AFFINITY PURIFICATION SEQUENCING

(71) Applicant: The Regents of the University of California, Oakland, CA (US)

(72) Inventors: Ronan O'Malley, Oakland, CA (US); Leo Baumgart, Oakland, CA (US)

(21) Appl. No.: 17/750,844

(22) Filed: May 23, 2022

Related U.S. Application Data
(60) Provisional application No. 63/191,553, filed on May 21, 2021.

(51) Int. Cl. C12N 15/10; C12Q 1/6806; C12Q 1/6874

(52) U.S. Cl. CPC C12N 15/1065; C12Q 1/6806; C12N 15/1006; C12Q 1/6874; C12Q 1/6855

(57) ABSTRACT

Described herein are affinity-labeled polypeptide compositions, such as affinity-labeled transcription factor compositions, and methods of using such compositions to evaluate interactions of the polypeptide with other molecules such as nucleic acids.

### CROSS-REFERENCE TO RELATED APPLICATIONS

This application claims priority benefit of U.S. Provisional Application No. 63/191,553, filed May 21, 2021, which is incorporated by reference for all purposes.

### STATEMENT AS TO RIGHTS TO INVENTIONS MADE UNDER FEDERALLY SPONSORED RESEARCH AND DEVELOPMENT

This invention was made with government support under Contract No. DE-AC02-OSCH11231 awarded by the U.S. Department of Energy. The Government has certain rights in this invention.

### BACKGROUND

Mapping transcription factor (TFs) to their target genes is fundamental to understanding how gene networks function. However, for most organisms no TF-gene interactions information is available. DNA affinity purification sequence (DAP-seq) combines advantages of in vivo and in vitro assays. DAP-seq directly measures TF binding in a native local genomic context in an in vitro TF-DNA binding assay that allows rapid generation of genome-wide binding site mapping for a large numbers of TFs, while capturing genomic DNA binding property that impacts binding in vivo (e.g., DNA methylation). The primary technical bottleneck to acquire such information is the significant effort involved in cloning and expressing tagged TFs used in in vitro assays.

### BRIEF SUMMARY

In one aspect, the present disclosure provides a method of affinity-labeling a polypeptide, e.g., a transcription factor, in an in vitro transcription and translation reaction to evaluate interactions of the polypeptide with other molecules of interest, such as cellular nucleic acids or proteins. The method employs a tRNA having an affinity-labeled amino acid, e.g., a lysine tRNA in which the lysine is linked to an affinity label, such as biotin. In some embodiments, a nucleic acid encoding a polypeptide of interest is amplified and transcribed in an in vitro transcription reaction. Labeled polypeptide is obtained by providing the tRNA loaded with labeled amino acid in an in vitro translation reaction. In some embodiments, the affinity-labeled polypeptide is a transcription factor. In some embodiments, an affinity-labeled transcription factor is employed to evaluate transcription factor binding sites. In some embodiments, an affinity-labeled transcription factor, e.g., a biotin-labeled transcription factor labeled at a subset of lysine residues with biotin, is used in DAP-seq (referred to herein as “biotin-DAP-seq” for convenience) to evaluate TF-genomic DNA binding interactions. In some embodiments, the binding moiety is biotin. One of skill understands that alternative binding moieties can also be used. The same approach used to immobilize TFs for DAP-seq can also be used for a low-cost high-throughput protein isolation for downstream characterization of interactions of the labeled protein with other molecules, e.g., protein-protein interactions, ligand binding, and/or structural analysis. In some embodiments, an affinity-labeled polypeptide generated as described herein is used in conjunction with other massively parallel sequence-based analyses. In some embodiments, an affinity-labeled polypeptide is used to evaluate binding of the polypeptide to RNA. In some embodiments, an affinity-labeled polypeptide is used to evaluate binding to synthetic nucleic acids, e.g., in a Systematic Evolution of Ligands by Exponential Enrichment (SELEX) method, e.g., for the identification of aptamers that bind the protein of interest.

In one embodiment, the disclosure provides a method, e.g., biotin-DAP-seq, in which labeled TF proteins are expressed from templates that are PCR amplified directly from genomic DNA or cDNA. Thus, for example, primers flanking a transcription factor gene are used to amplify the gene. Such amplification primers are designed to contain an appropriate promoter, such as a T7 promoter, and other required components for expression in an in vitro coupled transcription and translation reaction mixture. An affinity agent, such as biotin, is introduced into the TF polypeptide encoded by the TF gene during translation by including a tRNA loaded with affinity-labeled lysine, e.g., biotinylated lysine, in the translation reaction mixture. This results in incorporation of affinity moieties, e.g., biotin moieties, at a random subset of lysine codons within the protein sequence. The affinity moiety, e.g., biotin, allows for downstream affinity capture of TFs along with bound DNA sequences using a biotin binding agent, e.g., streptavidin, as a capture agent, e.g., using streptavidin-coated magnetic beads. Methods used to immobilize TFs for DAP-seq can also be used for a low-cost high-throughput protein isolation for downstream characterization of any kind of protein-protein interactions, ligand binding interactions, structural analysis, and other methods that evaluate interactions of a protein with another molecule. In some embodiments, the method described herein to affinity label proteins using charged tRNA to label a polypeptide in an in vitro translation reaction can be used in conjunction with massively parallel sequence analysis, including for example, massively parallel RNA sequence and synthetic DNA sequencing.

### BRIEF DESCRIPTION OF THE DRAWINGS

FIG. 1. Streamlined protein expression directly from PCR products amplified from genomic DNA circumvents the need for plasmid construction. Addition of tRNA loaded with biotinylated lysine results in incorporation of biotin tags at a random subset of positions that encode a lysine within the TF amino acid sequence. With this protocol an experiment can be completed in approximately 5 hours. See also FIG. 8.

FIG. 2A-2C. Overview of multiDAP experimental setup and example of resulting data. (2A) TFs were PCR amplified directly from E. coli or P. simiae genomic DNA and used as templates for in vitro protein expression, with biotin tags incorporated. (2B) Genomic DNA fragment libraries were prepared from 48 bacterial species, each with a unique molecular i5 barcode. All 48 libraries were pooled and distributed to TF protein plates. TFs bound to target DNA fragments and streptavidin-coated magnetic beads. After washing, remaining bound DNA fragments were PCR amplified using a unique i7 barcoded primer for each well before pooling and sequencing. (2C) After demultiplexing of sequence reads, alignment to genome revealed TF binding sites in each species evident as peaks in coverage plots. Coverage plots for a P. simiae TF Ps356 are shown in black across a 10,000 base pair window. Genes predicted to be regulated by Ps356 in each species are colored by predicted gene function and labels indicate BLAST amino acid identity to P. simiae orthologs.

FIG. 3A-3B. Quantification of TF target gene conservation across species reveals global patterns of conservation and evolution. Target gene similarity was quantified as the number of matching orthologs appearing in pairs of gene sets. P-values were determined by comparing to a mock set of target genes randomly selected from each genome for 10,000 iterations. Blue-to-red shades correspond to significance, with darkest red representing the most significant (p-value 1e-4). Gray-shaded vertical bars mark phylogenetic clades (top to bottom): Gammaproteobacteria, Betaproteobacteria, Alphaproteobacteria, Bacteroidetes, Gram-positive. (3A) For each E. coli TF (columns), the set of target genes in E. coli was compared to the set of target genes in each of the other 47 species (rows 1-48; refer to FIG. 4 for full genus and species). Closely related species in the Enterobacteria family are colored in blue. Starred TFs are discussed in text. (3B) The same analysis was applied to TFs and target gene sets from P. simiae. Closely related species in the Pseudomonas genus are colored in green.

FIG. 4A-4C. Selected TF target operons compared across species, exemplifying degrees of conversation and instances of rewiring. Predicted orthologs are color coded, with functional prediction from RefSeq annotations in legend. Solid colored genes are present in species from where TF originated (E. coli or P. simiae), outlined colored genes are absent. MultiDAP coverage tracks are shown in black underneath. (4A) The TF MraZ from E. coli regulates genes involved in cell division and cell wall synthesis and has highly conserved binding sites and operon structure across divergent clades. (4B) A TF from P. simiae (nicknamed Ps170) involved in sugar transport and metabolism is conserved in several species across the phylum Proteobacteria. In some species it targets multiple promoters, and the combination and organization of target genes varies. (4C) The TF ArsR from E. coli is an arsenic sensor that regulates genes for arsenic resistance and is conserved in species from the class Gammaproteobacteria, although the number of copies per genome varies. The TF is conserved as the first gene in the operon in all cases, but other downstream genes are variable.

FIG. 5A-5B. All-vs-all comparison of gene sets targeted by a given TF in each species reveals distinct clusters of conservation in different bacterial clades. (5A) The E. coli TF AscG provides an example of two distinct clusters of target genes: one cluster mainly limited to the Enterobacteria, and another extending across the genus Pseudomonas and into the class β-Proteobacteria. (5B) A closer inspection of the AscG target operons in the model organisms E. coli MG1655 and P. aeruginosa PAO1, along with predicted orthologs of these genes in other species, suggests that the TF's function has diverged between the two clusters. Genes are colored according to their orthogroup: E. coli genes and orthologs in solid colors, and those of P. aeruginosa with stripes. Functional predictions from RefSeq annotations are shown in legend. See also FIGS. 9 and 10.

FIG. 6A-6C. Combining multiDAP with phenotypic measurements enables establishment of functional regulons for a TF across multiple distant operons for multiple species. (6A) The E. coli autoregulator FucR is a known transcriptional activator which acts on an operon involved in fucose utilization. MultiDAP accurately predicts the target genes in E. coli, which is further supported by an observed fitness disadvantage conferred by gene knockouts in the corresponding operons. Both multiDAP and phenotypic measurement also support conservation of the FucR regulon in Klebsiella oxytoca. (6B) In the non-model organism P. simiae, multiDAP allows bundling of TFs and targets located in distant regions of the genome. TF Ps109 and genes found in two distant target operons play a role in 2′-deoxyinosine utilization, as evidenced by phenotypic measurements. The TF gene knockout confers a positive growth impact while the target gene knockouts display a negative impact, suggesting that the TF functions as a transcriptional repressor at the target promoters. (6C) MultiDAP results for TF Ps17 reveal a conserved TF and distant target gene found in 5 of the tested Pseudomonas species. Phenotype data shows a positive correlation between the phenotype of the TF and target gene when succinate is supplied as the sole carbon source, suggesting that Ps17 functions as a transcriptional activator and is involved in succinate utilization in all 5 species.

FIG. 7A-7D. Autoregulator TF binding sequence motifs mapped to promoter sequences reveal conserved promoter architecture. Logo representation of motifs is shown at top, coverage plots are in black, and motif locations are gray bars below. (7A) For the E. coli TF MraZ, the location of motifs relative to the gene start varies by species, but adjacent motifs are always spaced exactly 10 bases apart even in highly divergent species. Detailed view is shown for E. coli, P. simiae, and B. subtilis (left), and heatmap view is shown for all species (right). (7B) A TF from P. simiae (nicknamed Ps408) is limited to the genus Pseudomonas where it is found as a pair of motifs spaced 21 bases apart. (7C) The E. coli TF LysR binds to a single conserved motif located close to the gene start across various species within the phylum Proteobacteria. (7D) Autoregulator TF motifs are specifically enriched in the corresponding 100 bp promoter sequences of orthologs throughout metagenomic samples in the Integrated Microbial Genomes (IMG) database. See also FIGS. 11, 12, and 13.

FIG. 8A-8C. Related to FIG. 1. Validation of TF biotin tagging method and streptavidin-coated bead capture. Target genes annotated in RegulonDB were compared to those captured in the biotin DAP assay. We observe that binding sites for TFs with smaller numbers of published binding sites (panels 8A and 8B) are particularly well represented in our dataset. This may indicate that these TFs have properties that result in better performance in this modified DAPseq assay, such as stronger binding affinity or specificity.

FIG. 9. Related to FIG. 5. Comparison of the Pseudomonas aeruginosa PAO1 PtxS DNA binding sequence motif (top) to that of the Escherichia coli MG1655 TF AscG (bottom) shows high similarity (p-value=2e-7 as calculated by Tomtom).

FIG. 10. Related to FIG. 5. All-vs-all comparison of gene sets targeted by a given TF in each species reveals distinct clusters of conservation in different bacterial clades. In addition to AscG (as detailed in FIG. 5), multiple clusters of conserved gene target sets are apparent for 13 other E. coli TFs. Clusters tend to appear in the clades representing Enterobacteria (blue), Shewanella (gray), and Pseudomonas (green). This is expected, because within the 48 species, we sampled several more closely related species from within these three clades, while other lineages were sampled much more sparsely. In regions of sparse sampling, any existing clusters of conservation appear as a single red square, which precludes identification as a true cluster.

FIG. 11. Related to FIG. 7. New E. coli motifs from this work that are not represented in RegulonDB (n=66).

FIG. 12. Related to FIG. 7. E. coli motifs compared to known motifs in RegulonDB. We found good agreement between motifs computed from the multiDAP datasets and RegulonDB: 50 matches (86%) of 58 motifs represented in both datasets. Motifs were considered to be matches if the p-value was less than 0.01, as scored by Tomtom.

FIG. 13. Related to FIG. 7. The motif for E. coli TF YiaJ was recently described by Shimada et al (top), who established the TFs function in plant breakdown product utilization, and renamed it to PlaR. The motif compares closely to the motif computed from multiDAP in this work (bottom).

### DETAILED DESCRIPTION

In one aspect, the disclosure provides a method of incorporating an affinity moiety into a polypeptide, e.g., a transcription factor, during an in vitro transcription-translation reaction in which RNA is transcribed from a template in vitro, e.g., from a template from an amplification reaction such as PCR, and translated in a reaction in which a tRNA loaded with an amino acid coupled to the affinity moiety is included for incorporation into the polypeptide, thereby producing an affinity labeled-polypeptide. In some embodiments, the template for translation is a plasmid DNA, a viral nucleic acid, or other template provided in sufficient quantity to obtain sufficient affinity-labeled polypeptide for analysis of binding of the labeled polypeptide to nucleic acids, ligands, or other binding molecules. The binding moiety of the affinity-labeled polypeptide specifically binds to a binding partner, e.g., immobilized on a solid support such as a bead. The affinity-labeled polypeptide can be used to evaluate binding interactions of the polypeptide with various molecules of interest, including nucleic acids, such as DNA, RNA, and synthetic DNA generated by chemical synthesis; polypeptides, and ligands. In some embodiments, an affinity labeled-polypeptide is used in conjunction with a massively parallel sequencing analysis to identify polynucleotides that bind the captured affinity-labeled polypeptide.

### Amplification Reaction

In some embodiments, the method comprises a coupled in vitro transcription and in vitro translation reactions. A nucleic acid for use as a template for transcription can be any nucleic acid that encodes a polypeptide of interest, including genomic DNA, e.g., from an intronless gene, cDNA, chemically synthesized DNA, or a plasmid DNA template. In some embodiments, the template is an RNA strand in which an RNA-dependent RNA polymerase is employed to generate the corresponding RNA strand that is translated. In some embodiments, the template is generated using an amplification reaction. As used herein, “amplification” of a nucleic acid sequence has its usual meaning, and refers to in vitro techniques for enzymatically increasing the number of copies of a target sequence. The terms refers to both linear and exponential amplification. Amplification methods include both asymmetric methods in which the predominant product is single-stranded and conventional methods in which the predominant product is double-stranded. In typical embodiments, amplification comprises a PCR to obtain amplified products to serve as the template for transcription. Primers are typically designed to include an RNA polymerase binding site, such as an SP6, T7 or T3 binding site.

In some embodiments, the template is a plasmid DNA, which is provided in an amount sufficient to generate RNA that in turn is translated in vitro as described herein to generate affinity-labeled polypeptide. Thus, for example, in some embodiments, the template is a plasmid that comprises a promoter for transcription of RNA in vitro for translation in an in vitro translation reaction.

### In Vitro Translation Reaction

Translation systems for in vitro translation are known. In some embodiments, in vitro translation is coupled with in vitro transcription reaction. Translation systems include cell lysate translation systems, such as bacterial cell lysates, wheat germ lysates, and rabbit reticulocyte lysate translation systems. Such systems can be supplemented with additional components such as ATP, protease inhibitors, RNA polymerases, etc. In some embodiments, the in vitro translation systems is reconstituted from individual purified or partially purified components (e.g., a reconstituted E coli translation system using recombinant components as described in Shimizu et al. (2001) Nature Biotechnology 19, 751-755).

Amino Acid-Loaded tRNA

Any amino acid can be coupled to an affinity label, e.g., biotin, and loaded onto a corresponding tRNA. As used in this context, a “loaded” tRNA is used interchangeably with “precharged” tRNA to indicate that an amino acid is bound to its corresponding tRNA. In some embodiments, the amino acid coupled to the affinity label, e.g., biotin, has a reactive side chain. In some embodiments, the amino acid has a hydrophilic or charged side chain. In some embodiments, the amino acid is lysine, arginine, tyrosine, glutamate, aspartate or cysteine. In some embodiments, affinity-modified lysine is used to charge the corresponding tRNA. In some embodiments, the affinity label is biotin. In some embodiments, a precharged, affinity-labeled tRNA is provided in the in vitro translation reaction in an amount such that a desired percentage of labeled amino acid residues are incorporated into the product of the translation reaction. For example, in some embodiments, over 20%, e.g., from 20%-35%, or 20% to 50% of the residues of the amino acid selected for labeling are affinity-labeled in the translation product. For example, where biotin-lysine is employed as the affinity label, the proportion of biotinylated-lysine tRNA added to the translation reaction can be adjusted to obtain a translation product in which 20% or greater of the lysine residue, or 30% or greater of the lysine residues are affinity labeled.

In some embodiments, the tRNA anticodon site can target to a different codon than to the corresponding amino acid with which it is loaded. For example, a lysine-biotin loaded tRNA can encode an anticodon that targets a stop codon or a four-base non-natural codon to allow for incorporation of the lysine-biotin at a unique and specific location in the polypeptide.

Transfer RNAs charged with affinity-labelled amino acids, e.g., biotinylated lysine tRNA, are known in the art. For example, biotinylated lysine tRNA is available from Promega. In some instances, an affinity label is conjugated to an amino acid and subsequently loaded onto the corresponding tRNA. Such conjugation reactions are well known in the art. Examples include, but are not limited to amide coupling reaction, Michael addition reactions, hydrazone formation reactions and click chemistry cycloaddition reactions.

### Affinity Moiety

Affinity moieties and corresponding binding partners are well known in the art. In some embodiments, the affinity binding partner is immobilized on a solid support. The solid support can be any solid substrate, such as a well or other compartment, or a bead. In some embodiments the solid support is a bead, such as a magnetic bead. In some embodiments, the affinity agent is an aptamer, a hapten, a ligand, a dye, or a biotin binding moiety. Thus, for example, in some embodiments, the binding moiety is biotin and the affinity binding partner is streptavidin or avidin. In some embodiments, the binding moity/affinity binding partner is dithiobiotin/avidin, iminobiotin/avidin, dithiobiotin/succinilated avidin, iminobiotin/succinilated avidin, or biotin/succinilated avidin. In some embodiments, the binding moiety/binding partner comprise FITC/anti-FITC antibody, digoxigenin/anti-digoxigenin antibody, or a hapten or epitope and antibody that binds the hapten or epitope, dithiobiotin-avidin, iminobiotin-avidin, biotin-avidin, dithiobiotin-succinilated avidin, iminobiotin-succinilated avidin, biotin-streptavidin, and biotin-succinilated avidin. In some embodiments, the

### Methods Employing Affinity-Labeled Polypeptides

As explained above, in vitro transcription coupled with in vitro translation is employed to generate an affinity-labeled polypeptide. Such a labeled polypeptide can be used for any application to evaluate interactions of the polypeptide with other molecules, for evaluation of protein-protein interactions, protein-nucleic acid interactions, structure evaluation, and the like. In typical embodiments, affinity-labeled polypeptide is used in conjunction with a massively parallel sequencing methodology, for example to evaluate changes in polypeptide interactions with other molecules in different types of cells or cells subjected to different environment conditions.

In some embodiments, the methods described herein are employed to generate an affinity labeled transcription factor for evaluation of transcription factor binding to genomic DNA. In some embodiments, the affinity-labeled TF is employed in DAP-seq (O'Malley et al., Cell 165:1280-1292, 2016, which is incorporated by reference). In brief, affinity-labeled TF is incubated with genomic DNA obtained from an organism of interest. Fragmented genomic DNA is incubated with the affinity-labeled TF, and the affinity label is used to bind to its binding partner that is attached to a solid support. Genomic DNA bound to the TF can then be processed for sequencing, e.g., using any massively parallel sequencing technique, to determine TF binding motifs. The affinity-labeled TF can be incubated with genomic DNA either before or after the TF binds to its binding partner on the solid support. In DAP-Seq, genomic DNA fragments are typically amplified to incorporate adaptor sequence. As used herein an “adaptor sequence” comprises one or more functional sequences used for amplification, sequencing, identification of the genomic sample used in the analysis, and/or quantification. Thus, for examples, an adaptor sequence may comprise one or more of the following: a universal sequence employed for sequencing, a primer sequence, a cellular identification sequence, a unique molecular identifier (UMI) sequence, a sample identification sequence, and combinations thereof. An adaptor sequence may comprise double-stranded regions, single-stranded regions, or both. This disclosure is not limited to the type of adaptor sequences which could be used and a skilled artisan will recognize additional sequences which may be of use for library preparation and next generation sequencing.

In some embodiments, DAP-seq is employed in conjunction with other sequence-based analyses. Exemplary assays include RNA sequencing, including, but not limited to, sequencing of mRNA, and other RNA populations of interest such as miRNA, snRNA, lncRNA and the like. In some embodiments, DAP-Seq may be performed in conjunction with DNA bisulfite sequencing, e.g., methyl-seq to analyze methylation status, or ATAC-Seq.

In some embodiments, DAP-Seq can be employed in multiplex reactions in which affinity-labeled TFs are incubated with genomic DNA from a diversity of samples to identify TF binding motifs, e.g., TF binding motifs present in different organisms.

In some embodiments, the methods described herein are employed to generate an affinity-labeled protein for evaluation of binding to RNA. Thus for example, affinity-labeled polypeptides can be localized to beads and incubated with RNA obtained from cells. RNA bound to the immobilized polypeptide can then be processed for sequencing, e.g., using any massively parallel sequencing technique. In some embodiments, the affinity-labeled polypeptide is incubated with RNA after the polypeptide is captured on the solid support. The RNA can then be process for sequencing, e.g., a reverse transcriptase reaction, to generate DNA and incorporating adaptor sequences in a subsequent amplification reaction.

In some embodiments, an affinity-labeled polypeptide generated as described herein is incubated with a population of synthetic nucleic acid molecules, e.g., a library of sequences, to identify an aptamer that binds to the polypeptide. Nucleic acid aptamers are a class of small nucleic acid ligands that are composed of RNA or single-stranded DNA oligonucleotides folded into a three-dimensional structure that have high specificity and affinity for their targets. For example, SELEX technology can be used to obtain aptamers specific to the affinity labeled polypeptide. Nucleic acid aptamers can be produced by as chemical synthesis or in vitro transcription for RNA aptamers. Nucleic acid aptamers include DNA aptamers, RNA aptamers, XNA aptamers (nucleic acid aptamer comprising xeno nucleotides) and L-RNA aptamers.

As appreciated by one of skill in the art, any for the foregoing methods can be performed in a multiplex reaction analyzing different populations of nucleic acid molecules, e.g., nucleic acids samples obtained from different types of cells, nucleic acid samples from single cells for analysis of nucleic acid profiles of single cells from a population of cells.

As noted above and as appreciated by one of skill in the art, an affinity-labeled polypeptide can also be used in various analyses for identification and characterization of polypeptides interactions with any number of molecules, including other polypeptides, polynucleotides, carbohydrates, glyoclipids, and any other molecule of interest. Such analyses can be conducted in combination with other high throughput, massively parallel sequencing analyses, as illustrated above.

In a further aspect, the disclosure provides kits and reagents for analyzing binding interactions of an affinity-labeled polypeptide with other molecules. In some embodiments, a kit can comprise sequencing adaptors, reagents for in vitro translation to label the polypeptide with an affinity moiety, and optionally, other reagents to perform massively parallel sequencing.

### EXAMPLES

### Example 1. Affinity Labeling of TF Polypeptides In Vitro Using Biotinylated-Lysine tRNA

Understanding the interactions between TFs, their binding sites, and the collection of target genes they regulate is key to our ability to model transcriptional programs and ultimately engineer them. However, large-scale decoding of these interactions is currently limited to a small set of model organisms, in part because of the limitations posed by existing technologies. In vivo methods such as ChIP-seq¹⁻⁴ can capture TF binding in a physiologically relevant state, but are difficult to scale up to match the hundreds to thousands of TFs found in a single organism. In contrast, in vitro methods such as protein binding microarrays (PBMs)⁵ and systematic evolution of ligands by exponential enrichment (SELEX)⁶⁻⁸ can be leveraged at large scales. However, most in vitro methods rely on indirect characterization of binding sites by identifying TF binding motifs using synthetic short DNA sequence pools, followed by scanning for these motifs in the reference genome to predict TF binding sites. As a result, these in vitro assays are unable to capture effects of native genomic context including DNA shape, chemical modifications, and conserved local cis-element architecuture that can have a large impact on TF binding specificity.

DNA affinity purification sequencing (DAP-seq), the method we developed in 2016,⁹ uniquely combines the advantages of in vivo and in vitro assays. Similar to ChIP-seq, DAP-seq directly measures TF binding in native local genomic contexts, and can be scaled up to comprehensively assay all TFs within a species, as demonstrated previously with Arabidopsis. To achieve this, DAP-seq leverages in vitro expressed and affinity-purified TFs to capture binding events with fragmented native genomic DNA (gDNA), followed by high-throughput sequencing.¹⁰ DAP-seq has proven to be an effective method to study TF binding sites in a variety of model organisms¹¹⁻¹³ and the resulting large-scale datasets have been central to a variety of approaches for understanding gene regulation.¹⁴⁻¹⁶

One limitation to DAP-seq, as well as all other existing TF binding assays, is the significant upfront investment required to purify each TF of interest. This is the major bottleneck for all high-throughput TF DNA binding techniques, and the primary restriction on the total number of TFs that can be assayed. In the original DAP-seq method, TF proteins are expressed in vitro from E. coli plasmid templates, which allows fusion of the TF coding sequence with an affinity tag that is required for the pulldown of the expressed TF and the DNA sequences it binds to. This limits widespread application to non-model organisms for which pre-existing TF plasmid collections are not available, and in particular to microbial studies where short generation times and high mutation rates have generated a diversity of TFs too vast to be practically surveyed using a plasmid-based approach. In addition, the original DAP-seq method only enables mapping gDNA binding properties in a single genome at a time. The relationships between TFs, their binding sites, and target genes are known to be conserved sometimes over incredibly long periods of time, and have been shown to be a predictor of conserved biological functions.^{17,18} Therefore, a broader understanding of how TF binding sites and target genes evolve across phylogenetically relevant sets of species will be of great value to reveal the conservation, evolution, and the function of TF-target gene pathways, of which our current understanding is very limited.

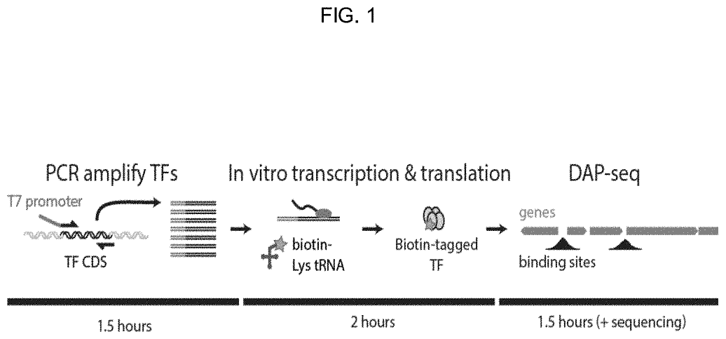

This example illustrates the production of biotin-labeled TFs using biotinylated lysine tRNA. A schematic is provided in FIG. 1 that illustrates Biotin-DAP-seq as an example of using biotinylated lysine-loaded tRNA to label polypepitdes. Biotin-DAP-seq is a streamlined clone-free workflow in which tagged TF proteins are expressed from templates that are PCR amplified directly from genomic DNA. First, we designed primers flanking the gene of interest. The primers contained a T7 promoter and other required components for expression with a commercial in vitro coupled transcription and translation mix, but did not contain an affinity tag sequence. Instead, a biotin tag was introduced directly during translation by spiking in a tRNA loaded with biotinylated lysine.¹⁹ This resulted in incorporation of biotin tags at a random subset of lysine codons within the protein sequence. This biotin-tag allowed for downstream affinity capture of TFs along with bound DNA sequences, using streptavidin-coated Here we present two new complementary methods, biotin DAP-seq and multiplexed DAP-seq (multiDAP), that together enable inexpensive and high-throughput surveys of binding sites for all TFs within a species and simultaneous mapping of conserved binding sites across a broad phylogenetic set of relevant species.

### TF PCR Amplification

Primers specific to each transcription factor were designed against the first and last 20-24 bases of the corresponding coding sequence. All non-standard start codons were switched to ATG. In each forward primer, a 5′ constant region was introduced immediately upstream of the sequence annealing to the start of the coding sequence, containing a T7 polymerase promoter and Kozak sequence. In each reverse primer, a 5′ sequence of 30×T was introduced to mimic a poly-A tail and facilitate protein expression in eukaryotic in vitro systems. These primers were used to amplify transcription factor coding sequences directly from the genomic DNA using KAPA HiFi 2×PCR master mix with the following conditions: Ta=60° C., 2 minute extension time at 72° C., total reaction volume=50 uL, for 24 cycles of PCR. PCR products were checked for amplification specificity using an Agilent 2200 TapeStation instrument. PCR products were purified using Omega Mag-Bind TotalPure NGS SPRI beads and eluted in 12 μL Tris-HCl buffer pH=8, yielding a DNA concentration of approximately 20-200 ng/μL.

### In-Vitro Protein Synthesis

TF proteins were expressed in vitro in 96-well microtiter plates using Promega TnT T7 Quick for PCR DNA following the manufacturer's protocol. For each 50 μL reaction, we use 5 μL of purified TF PCR product for a total of 250-1000 ng template. Negative control wells were included containing mock PCR product, where the PCR was performed with water in place of primers. In order to produce biotin-tagged TF proteins that can later be purified using streptavidin-coated beads, we also spiked in 4 μL of Promega Transcend tRNA to each 50 μL reaction. After combining all components at 4° C., the mixture was incubated at 30° C. overnight (12-18 hours).

The following example illustrates the use of the biotin-labeled TF proteins in DAP-SEQ.

### Example 2. Biotin-DAP-Seq

We investigated 354 transcription factors in 48 bacterial genomes, and generated 17,000 high quality TF binding site maps. This unprecedented rich dataset revealed themes of ancient conservation as well as rapid evolution of gene regulatory modules. We observed various patterns of evolution and regulatory rewiring, where the TF's sensing and regulatory role is maintained while the arrangement and identity of target genes diverges. Such regulatory rewirings execute analogous functions in some cases, while in others appear to have been repurposed for entirely new functions. We also integrated existing phenotypic information, established novel functional regulatory modules, and defined new pathways. Finally, we identified 242 new TF DNA binding motifs, yielding a 70% increase of characterized TF motifs in Escherichia coli, and annotations of TF motifs in Pseudomonas simiae for the first time. Integrative analyses of the TF DNA motifs across bacterial genomes revealed deep conservation in gene promoter architecture. Our methods are highly versatile for rapid characterization of gene pathways across any organisms, enabling direct annotation and dissection of regulatory pathways and laying the foundation for modeling and designing synthetic regulatory networks.

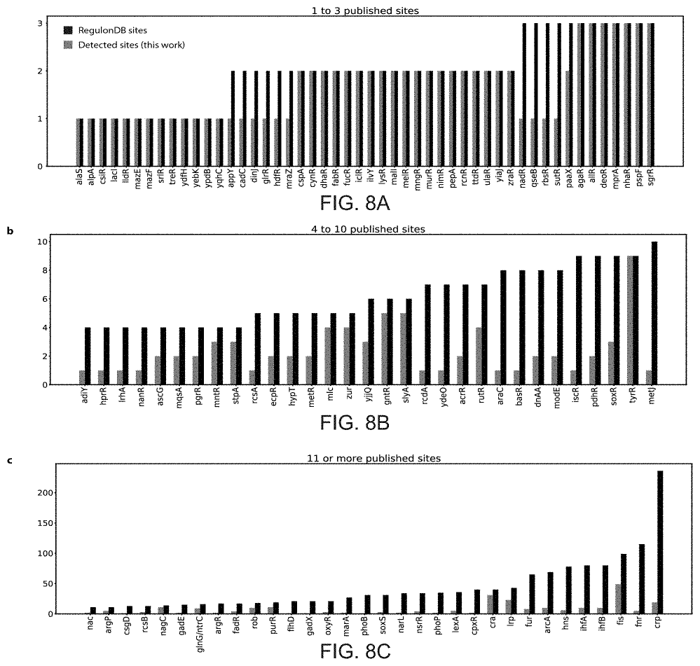

We validated this new streamlined TF expression approach (Example 1) using a test set of 216 known Escherichia coli TFs and observed one or more putative binding sites in at least one of two trials for 125 TFs (58% successful of 216 total). We then compared our results to previously described TF binding sites published in RegulonDB²⁰ (FIG. 8). Of these 125 TFs, we detected at least one published binding site for 113 TFs (90% of successful), at least half of sites for 64 TFs (57%), and all sites for 40 TFs (32%). These results demonstrate that this biotin-tagging approach does not prevent proper TF protein folding and binding, and allows detection of known functional TF binding sites. By eliminating the need for plasmid construction, we reduced the time required to produce a species-wide DAP-seq dataset from months to days, and the total reagent cost for the DAP-seq assay by more than half.

The streamlined biotin-DAP-seq is particularly suited to studying non-model organisms. We demonstrated this by mapping TF binding sites in Pseudomonas simiae,²¹ an emerging model for plant-commensal microbes that currently has no available TF binding site annotations.^{22,23} We compiled a comprehensive set of 567 putative P. simiae TFs by combining three different predicted gene annotations from GenBank²⁴, RefSeq²⁵, and IMG.²⁶ We initially screened the entire set of 567 TFs in two replicate DAP-seq experiments, of which 138 (24%) were successful as defined by at least one peak observed in both replicates. The lower overall success rate compared to the well characterized E. coli TFs is not surprising, as we screened any gene with predicted DNA-binding activity, many of which may not be functional TFs. We chose to use this set of 138 P. simiae TFs for further characterization.

Multiplexed TF Mapping: multiDAP

In parallel, we developed multiDAP, a method that allows mapping TF binding sites in multiple genomes simultaneously. The central concept of multiDAP is to leverage the fact that in the original DAP-seq assay the immobilized TF binding sites were not saturated, and by using a pool of gDNA samples from different species or strains we can directly map TF binding sites across a diverse array of organisms.

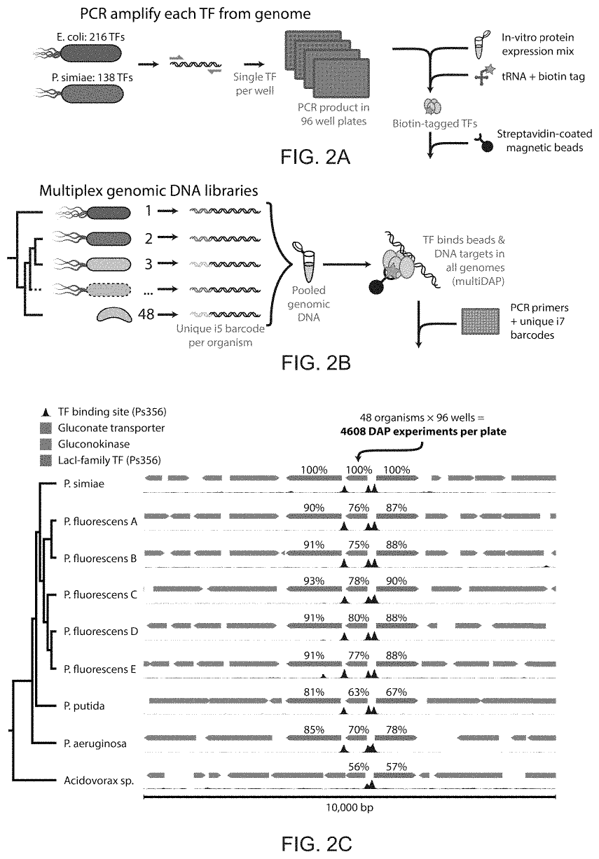

For our TF set we used a total of 354 TFs, including the 138 P. simiae TFs that were successful in the preceding biotin DAP-seq screen, and the entire 216 E. coli TF set regardless of success or failure in the preceding screen (FIG. 2a). Next we prepared genomic DNA fragment libraries from each of 48 bacterial genomes, each marked with a unique molecular barcode (FIG. 2b). We selected the set of 48 bacterial species to cover a large evolutionary distance, with a higher proportion of close relatives of E. coli and P. simiae for higher resolution of local conservation and variation. We pooled all 48 barcoded genomic DNA fragment libraries, and distributed this pool equally to each well of the microtiter plate containing bead-immobilized TF proteins. After several washing steps, the bound gDNA fraction was eluted and amplified by PCR using a set of uniquely barcoded PCR primers to mark the identity of each well and the corresponding TF. At this point all samples were pooled together for sequencing.

Based on the combination of molecular barcodes from each sequencing read, the dataset was computationally de-multiplexed to yield the equivalent of one DAPseq dataset per TF per organism. After alignment to the corresponding genomes, regions that contain TF binding sites were apparent as peaks, resulting from the pileup of DNA fragments that are bound by the TF. By mapping the binding of the 354 TFs from E. coli and P. simiae across the set of 48 bacterial genomes, we produced a combinatorial dataset equivalent to 17,000 DAP-seq experiments. This dataset allowed direct comparison across divergent bacterial species to reveal conserved patterns and evolution of TF binding at orthologous genes (FIG. 2c). We recovered high quality binding information for 113 of the 138 (82%) P. simiae TFs that were successful in the original single organism screen, and 107 of 216 (50%) of the E. coli TFs, a similar recovery rate to the 58% successful in the single organism screen. The difference between these single organism and multiDAP success rates may be in part because the single organism successes were the union of two replicates while the multiDAP was a single pass experiment.

### Evolutionary Conservation of Tf Targets

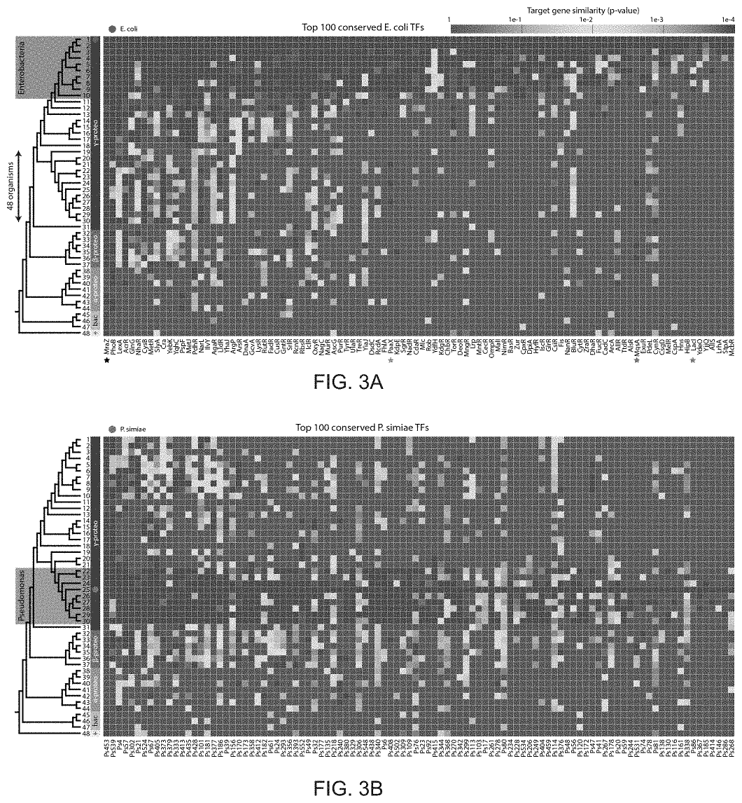

Using the resulting multiDAP dataset, we quantified the degree of TF target conservation across the 48 bacterial strains and species. Given that transcription factors often bind in the promoter region directly upstream of genes in bacteria,²⁷ we assigned each peak to the predicted operon(s) that are directly adjacent to and oriented away from the peak. Thus for a given TF, we compiled a set of genes that we predict may be regulated by the TF in each of the organisms. We then calculated a target gene similarity score by comparing the sets of target genes across organisms. We first grouped all protein-coding genes from all 48 species into groups of putative orthologs (orthogroups).²⁸ Next, we quantified TF target conservation by comparing the set of orthogroups targeted in the species from where the TF itself originated (either E. coli or P. simiae) with those targeted in each of the remaining 47 organisms. The results of this analysis give a global view of TF target gene similarity in divergent bacteria for both TFs from E. coli (FIG. 3a) and TFs from P. simiae (FIG. 3b). Strong matches suggest conserved gene regulation by the corresponding TF ortholog, and may serve as attractive choices for future studies and in vivo characterization.

We observed that while some TFs and their targets appear to be confined to a small subset of species, others are highly conserved across large evolutionary distances. As may be expected, there appears to be a general trend where the majority of TF-target relationships from E. coli are well conserved within the closely related Enterobacteria clade. A similar degree of conservation is apparent when considering TFs from P. simiae within the Pseudomonas clade. One striking feature is the high degree of conservation of several TF targets across clades that diverged long ago. For example, the most highly conserved TF targets appear to be those of the MraZ transcriptional repressor from E. coli which regulates its own expression as well as genes involved in cell division and cell wall synthesis (FIG. 3a, left-most column marked by black star). This result is supported by previous studies, which have shown MraZ to be a highly conserved regulator in diverse bacterial species.^{29,30} Remarkably, our results indicate that the underlying DNA binding sequence is so well conserved that the E. coli MraZ protein is able to bind specifically to the promoter of the mraZ ortholog in Bacillus subtilis, a Gram-positive bacterium which diverged approximately 2 billion years ago.³¹ We find several additional TFs with apparent conservation far beyond the E. coli clade, many of which are known to be involved in diverse processes central to bacterial survival and replication, including PhoB (inorganic phosphate metabolism), LexA (response to DNA damage), AcrR (multidrug resistance), and GlnG/NtrC (nitrogen metabolism).

In contrast to these highly conserved features, we also observe evidence of regulatory changes at the sub-species level. To test the ability to accurately discriminate small genetic differences in gene regulation, we included two very closely related strains of E. coli (FIG. 3a, top 2 rows). As expected, we find that the target genes of almost all E. coli TFs are conserved between the two strains, with a single notable exception for the Lad repressor protein (FIG. 3a, column marked by orange star). This is consistent with the deletion of the Lad binding site upstream of the lacZ gene, which is among the small set of documented genetic differences between these two strains of E. coli.³²

A third category of features appear to be less conserved even in closely related species, yet are scattered across larger evolutionary distances. For example, the E. coli MqsA regulator of the mqsA/mqsR toxin/antitoxin system is found sporadically throughout the phylum Proteobacteria: E. coli and Pseudomonas putida in the class Gammaproteobacteria, as well as Ralstonia sp. and Herbaspirillum seropedicae in the class Betaproteobacteria (FIG. 3a, column marked by green star). Similarly, in the case of the the E. coli TF PaaX, binding sites upstream of genes involved in phenylacetic acid utilization are conserved in seven of the ten Enterobacteria, but also in a subset of the more distantly related Pseudomonas and Marinobacter genuses (FIG. 3a, column marked by gray star).

### Operon Shuffling and Genetic Rewiring

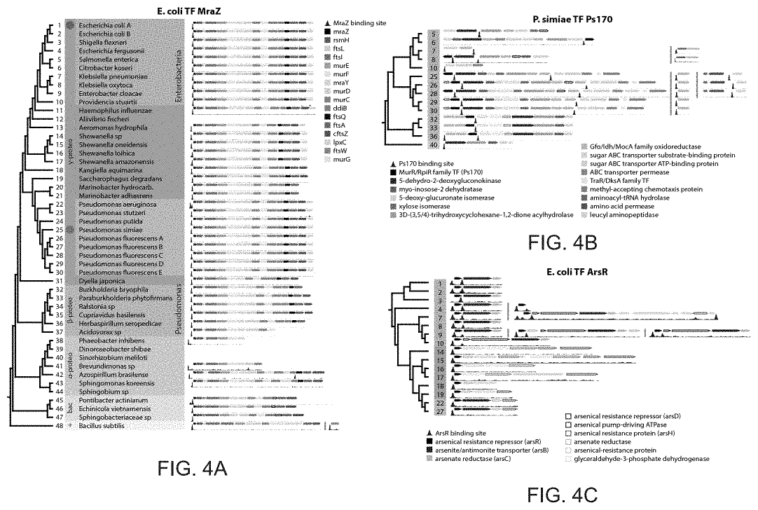

While the global analysis gives general insights into conserved binding features, closer inspection of specific TF targets offer examples of evolution within target operon structure. The E. coli autoregulator MraZ shows a strongly conserved operon structure, with only small differences in the gene content and their arrangement in operons in even the most distantly related species (FIG. 4a). We also observe more complex arrangements, exemplified by a TF from P. simiae that is conserved in species throughout the phylum Proteobacteria, where it targets multiple sites spread across distant regions of the genome (FIG. 4b). This TF likely regulates genes involved in several pathways related to sugar import and metabolism, and these genes are organized in operons showing a high frequency of re-shuffling.

An extreme example of divergence is seen in the case of the E. coli arsenic resistance regulator, ArsR, which is limited to bacteria sampled from the class Gammaproteobacteria (FIG. 4c). In E. coli, ArsR regulates a set of genes that detoxify arsenic through the combined action of an arsenic exporter (ArsB) and a reductase (ArsC).³³ However in four species of Shewanella the ArsR target operon does not contain any orthologs of these genes, except ArsR itself. Instead they encode distinct arsenic resistance proteins as well as an enzyme predicted to be a glyceraldehyde-3-phosphate dehydrogenase, which is also known to be involved in alternative pathways for arsenic detoxification.³⁴ These examples illustrate various patterns of evolution and rewiring, where the TF's sensing and regulatory role is maintained while the arrangement and identity of target genes diverges.

### Functional Repurposing of TFs

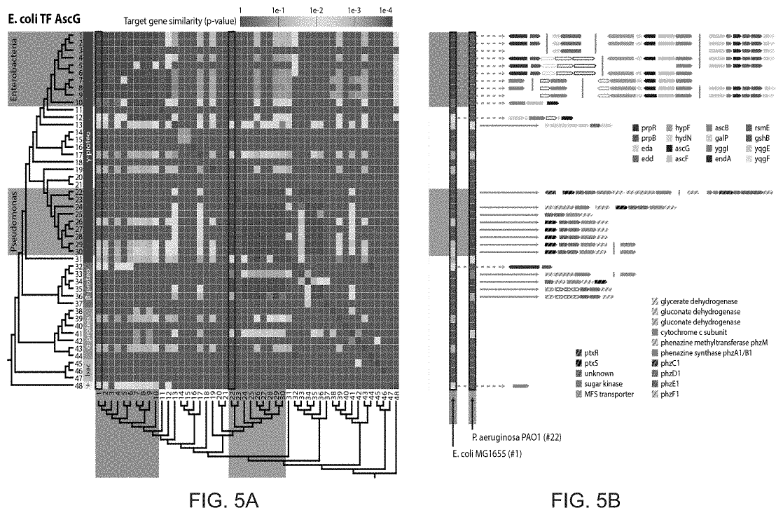

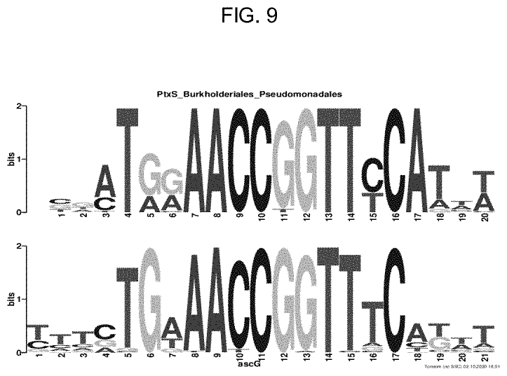

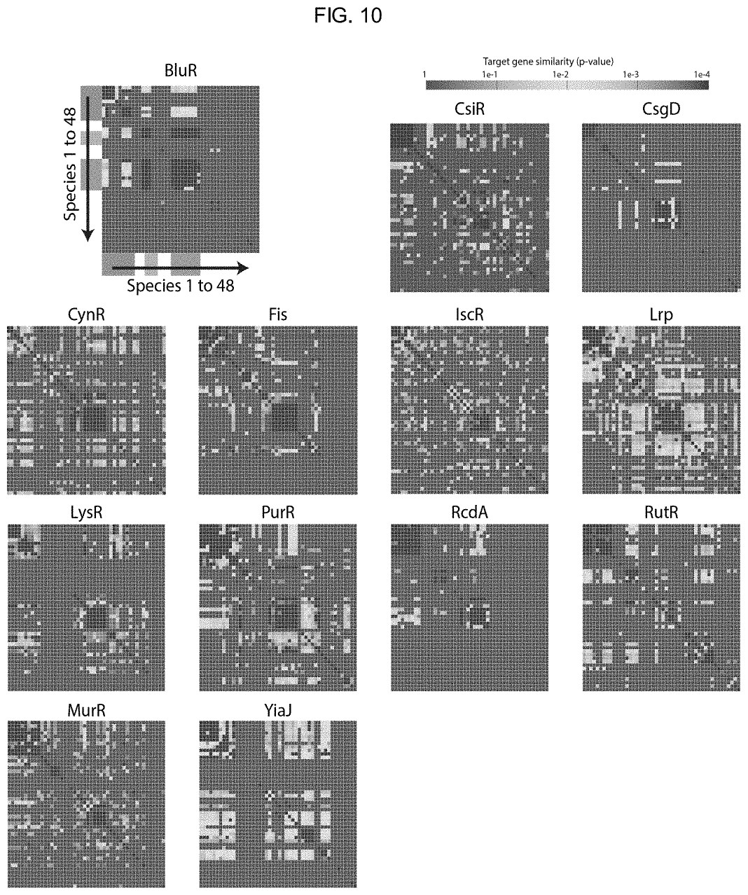

Having observed evidence of rewiring in TF targets, we next examined the dataset for examples of TFs that had diverged to take on entirely new functions within different bacterial clades. In order to identify clusters of conservation, we analyzed each TF individually and compared target gene sets of the 48 species to each other. This revealed clusters of conserved target genes and operons that are not found in the species from which the TFs originate (either E. coli or P. simiae). For example in E. coli, the TF AscG regulates genes involved in β-glucoside sugar and propionate utilization.^{35,36} While the majority of this E. coli regulon appears to be conserved throughout the Enterobacteria and in a few scattered organisms outside this clade, a second separate cluster extends across the genus Pseudomonas and into the class β-Proteobacteria (FIG. 5a). This second cluster includes the model organism Pseudomonas aeruginosa, where it targets genes that are predicted to function in glycerate/gluconate metabolism as well as synthesis of phenazines, pigmented compounds that have been implicated in antimicrobial resistance (FIG. 5b).³⁷ A strong binding site just upstream of the P. aeruginosa ptxS gene, a known autoregulator TF,³⁸ raises the possibility that PtxS serves as the counterpart in P. aeruginosa to AscG in E. coli. Indeed, both AscG and PtxS belong to the LacI/GalR protein family, and although they share only 24% amino acid identity (with 48% similarity and 98% coverage) their DNA binding sequence motifs are remarkably similar (p-value=2e-7, FIG. 9). In addition to AscG, we also observed 13 other examples of E. coli TFs and 25 P. simiae TFs that display evidence of broad evolutionary divergence, with distinct clusters of conservation in different lineages (FIG. 10).

### Functional Annotation of TFs and Regulons

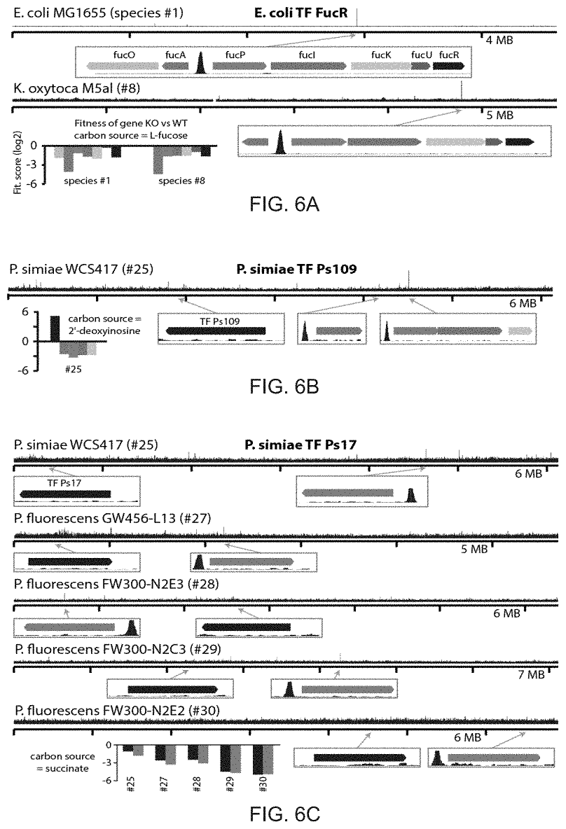

Our multiDAP species target set overlapped with 33 species utilized in a previous study designed to measure the fitness costs of gene knockouts on a range of conditional challenges including limiting carbon and nitrogen sources.²³ To investigate how the multiDAP and phenotype datasets can complement each other we initially identified a simple and well-characterized example from E. coli, FucR. In response to environmental sources of fucose, E. coli FucR activates genes involved in fucose import and degradation, as well as as the expression of FucR itself (i.e. autoregulation).³⁹ Disruption of fucR or other genes in the fuc operon resulted in a similar growth deficit. Similarly, in Klebsiella oxytoca when the ortholog of fucR or genes in its operon were knocked out, a fucose-dependent growth defect was observed. In both E. coli and K. oxytoca the binding sites predicted by the E. coli FucR multiDAP experiment correctly identified the TF and target genes for fucose sensing and metabolism (FIG. 6a).

We then investigated the non-model species P. simiae, where we used the multiDAP data in conjunction with phenotype information to establish functional relationships when transcription factors and target genes are at distant locations in the genomes. For example, in the muliDAP we observed that the P. simiae TF Ps109 appears to regulate genes at two distantly located promoters. While the TF knockout confers a growth advantage when 2′-deoxyinosine is the sole carbon source, knockouts of all four regulated genes show a growth disadvantage. The multiDAP binding information allows bundling of this phenotypic information to establish a functional regulatory model, with TF Ps109 acting as a transcriptional repressor at two distant operons involved in 2′-deoxyinosine utilization (FIG. 6b).

A third example, TF Ps17, shows how multiDAP allows bundling of phenotype information both across distant genome locations as well as across species, indicating Ps17's conserved function in succinate utilization (FIG. 6c). The existing annotations for both the transcription factor (Fis family transcriptional regulator) and target gene (C4-dicarboxylate transporter) are likely too generic to have informed a clear relationship between these TFs and their targets, demonstrating the value of multiDAP to identify regulatory modules within and across species.

### Motifs and Promoter Architecture

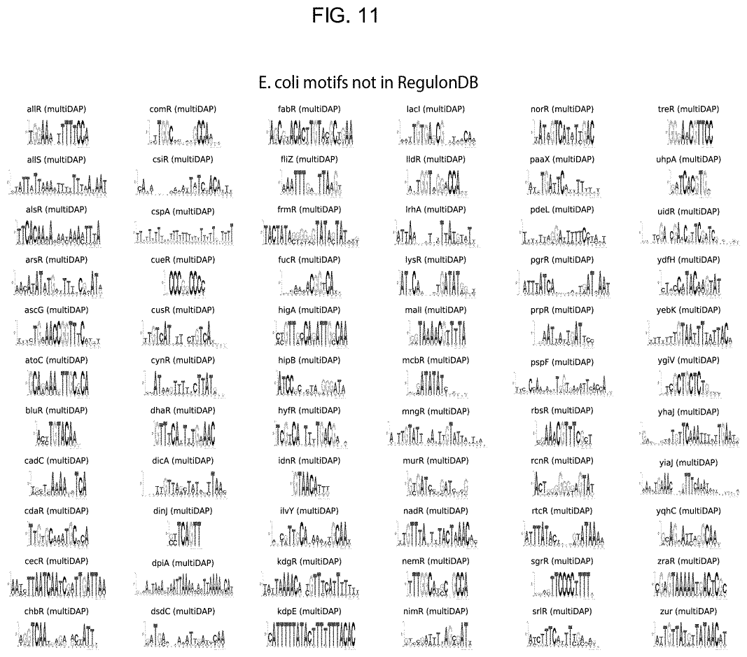

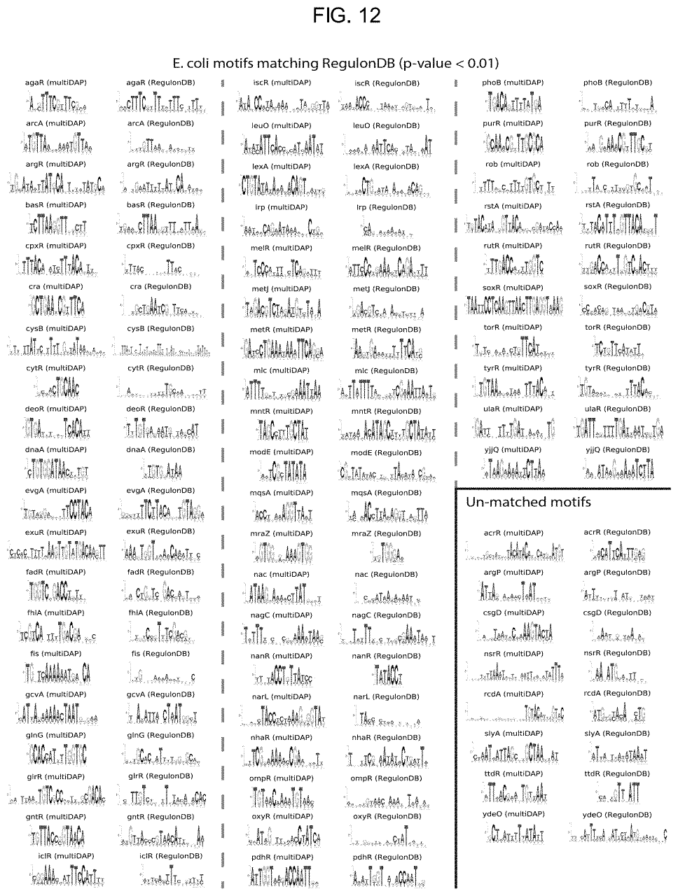

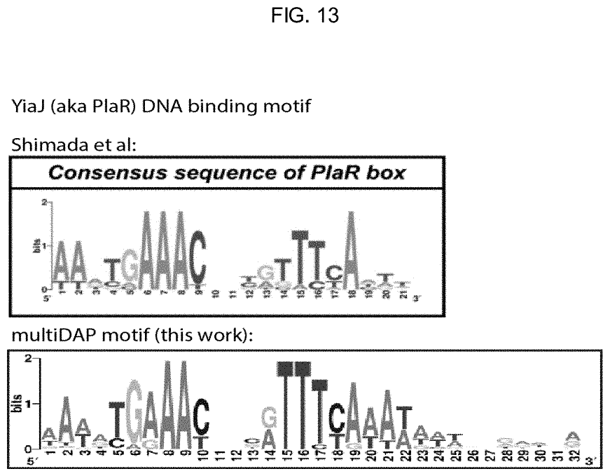

One challenge when studying bacterial transcription factor binding sequence motifs is that many TFs only bind strongly to a small number of sites in an entire genome, which can make it difficult to confidently identify a binding sequence motif. However, by assaying 48 microbial genomes in a single multiDAP experiment, the total number of binding sites for each TF in this dataset is multiplied by the number of species containing TF binding sites. We were able to call a high quality motif for 124 TFs from E. coli, 66 of which are not represented in RegulonDB (FIG. 11).²⁰ For the remaining 58 that are in RegulonDB, we found good agreement between the database and the motifs derived from this multiDAP dataset (50 matches (86%) of 58 motifs, each with p-value <0.01, FIG. 12). We also found good agreement between our motif for the E. coli TF YiaJ and the motif published in a recent study which established the function of this TF as involved in plant breakdown product utilization (FIG. 13).⁴⁰ The union between 93 known motifs and those newly reported here now provides a total of 158 E. coli motifs, a 70% increase over what was previously known for this model bacterium. As no published motifs exist for P. simiae, all 118 reported here are new motifs. These results demonstrate that multiDAP experiments offer an expedient and cost-effective method for generating high quality TF binding sequence motifs.

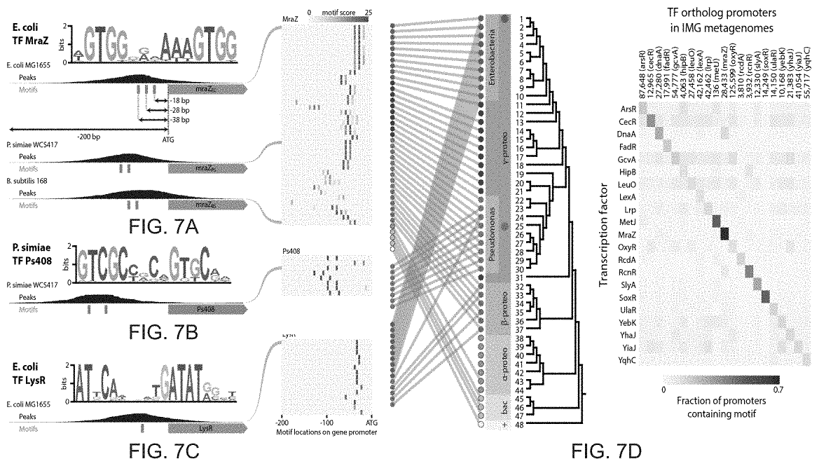

We applied these motifs to explore conservation and variation in TF binding site architecture in the promoters of orthologous genes. We mapped motifs back to promoter sequences to identify the exact location and orientation of binding sites in promoters across species. Auto-regulating TFs serve as a particularly tractable set, because there is less ambiguity in identifying the corresponding promoters to compare from each genome. We observe a variety of patterns, some of which are well conserved across divergent species. For some TFs such as MraZ, we observe closely spaced clusters of multiple motifs with variability in the number and strength of motifs (FIG. 7a), but where the motif orientation is always conserved and spacing between individual motifs within a cluster is always exactly 10 base pairs. Another common pattern is exemplified by TF Ps408, which is limited to species in the Pseudomonas clade and always appears as a doublet with two strong motifs and a 21 base pair gap in the middle (FIG. 7b). Yet others, such as LysR from E. coli have a single strong motif located close to the beginning of the coding sequence (FIG. 7c). Conserved promoter architecture likely reflects attributes of the TF proteins themselves, including size and shape, ability to form multimers, and protein-protein interactions with other TFs and sigma factors.

Beyond revealing conserved promoter architecture in known gene targets, TF binding sequence motifs can also aid in identifying previously unknown regulatory targets. We expanded our analysis beyond the 48 bacterial species by searching for TF orthologs in all metagenome assembled genomes in the Integrated Microbial Genomes (IMG) database,²⁶ based on amino acid sequence identity. We identified approximately 1.25M possible orthologs, of which >170 k showed evidence of conserved auto-regulation where TF motifs are enriched in their respective promoters (FIG. 7d). This demonstrates that the presence of motifs coupled with sequence similarity to previously identified target genes can provide further evidence to support protein function predictions in species beyond those tested directly.

### Summary of Biotin-DAP-Seq Example

In non-model organisms and metagenomes, a genome sequence provides a wealth of information about gene content and allows prediction of gene function based on similarity to known proteins, however the function of intergenic sequences remains difficult to annotate. In this work, we used multiDAP to identify TF binding sites in 48 diverse bacterial species as well as define 242 high quality binding site motifs for TFs from E. coli and P. simiae, most of which have not been previously described. This multiDAP dataset illustrates patterns of evolutionary rewiring and TF repurposing and defines new gene regulatory modules that are conserved across multiple species. The motifs described here can also be valuable in studying promoter architecture, functionally annotating metagenomic sequences, and designing novel synthetic promoters with desired regulatory properties. Beyond serving as a starting point for future characterization, these results also provide a blueprint for further multiDAP experiments. The new biotin DAP-seq approach facilitates rapid and inexpensive production of expressed TFs, while multiDAP allows analysis of many genomes simultaneously, thereby enriching the biological information extracted from each experiment. These two new techniques can be applied independently or in conjunction for large-scale studies, to begin mapping transcriptional regulatory networks and annotating functional gene regulatory modules across all kingdoms of life.

### Methods Employed for Example 2

### Fragment Library Construction

Genomic DNA from each organism was first sheared using ultrasonic shearing (Covaris LE220-plus) using the following settings: peak power=450W, duty factor=30%, cycles/burst=200. DNA was sheared to an average size of 75 bp in Tris-HCl buffer (pH=8) and applied in multiple cycles of 30 minutes each for a total of 60-90 minutes, allowing time for the water bath to cool between cycles such that the maximum temperature of the samples did not exceed 15° C. After shearing, up to 1 μg of each genomic DNA sample was used to prepare fragment libraries using the KAPA HyperPrep kit and standard manufacturer's protocol. During the adapter ligation step, custom annealed Y-adapters were introduced at a concentration of 15 pM (5 μL adapters in a reaction volume of 110 μL, final adapter concentration=0.7 pM). These custom adapters were prepared by annealing a full-length i5 index adapter with a stub i7 adapter. Ligated libraries were amplified for 8-10 cycles using primers P1 and P2 stub. Oligonucleotide and barcodes as well as strains used in this work are detailed in the supplementary information.

### MultiDAP Assay

ThermoFisher Dynabeads MyOne Streptavidin T1 were pelleted on a magnetic rack, washed 4× in PBS pH=7.4+0.1% v/v Tween20 and resuspended in an equal volume of this buffer. For each reaction, the following were combined in a mastermix (volumes given are per well/reaction): 15 μL resuspended beads, 1 μg salmon sperm DNA, and 1 ng amplified DNA fragment library from each organism, and topped off with PBS pH=7.4+0.1% v/v Tween20 to a final volume of 50 μL. Mastermix volume was scaled up for 384 samples. Subsequent steps were carried out in 96-well plates using a Hamilton Vantage liquid handler.

The bead+library master mix was aliquoted into each well of a 96-well plate, topped off with 50 μL PBS pH=7.4 and stamped into the plates containing the expressed TF proteins. Plates were incubated for 1 hour at room temperature, with gentle pipet mixing every 2 minutes to keep beads from settling.

After incubation, beads were pelleted and washed 4× with PBS pH=7.4+0.1% v/v Tween20, then resuspended in 10 μL i7 index primers (reference supplementary table) diluted to a final concentration of 1 uM each in Tris-HCl pH=8. An additional 10 μL KAPA HiFi 2×PCR master mix was added to each well. Plates were sealed, vortexed, centrifuged, and placed directly onto a thermocycler running the following program: an initial elution/denaturation step of 98° C. for 10 min, followed by 10 cycles of 98° C. for 10 sec, 60° C. for 30 sec, and 72° C. for 30 sec, with a final extension time of 72° C. for 1 min then a hold at to 4° C. Finished PCRs were pooled across each 96-well plate, using 10 μL from each well and purified using a 1.4× Ampure bead ratio, followed by elution in 30 μL Tris-HCl pH=8. In subsequent experiments we found that including an additional gel purification step to remove primer and adapter dimer carry-over is helpful to reduce issues related to index hopping on Illumina sequencers.

### Sequencing

Pooled sequencing libraries were quantified by qPCR and sequenced on NovaSeq 6000 S4 Flowcell to target ˜1 M reads for each of the 36,846 barcode pairs (384 TFs/wells×48 genomes). Libraries were de-multiplexed, adapter trimmed, and quality filtered using BBTools⁴¹.

### MultiDAP Analysis Pipeline

Analysis scripts are described in brief below. Code is available upon request. Each library was subsampled to at most 1 M fragments, aligned against the corresponding reference genome using Bowtie2⁴² and quality filtered with samtools.⁴³ Coverage plots were generated using deeptools.⁴⁴ Peaks were called using MACS2.⁴⁵ We observed a significant degree of index hopping, which resulted in cases of leak-through of signal between i7 barcodes. This was addressed using a custom script to identify and filter out cases of overlapping peaks for libraries that had been loaded on the same NovaSeq flow cell lane. This approach was also used to remove any peaks that are present at similar strengths in negative control wells (i.e. wells with mock expressed TF proteins). Target gene assignment for each peak was done using the reference annotation (gff3) and bedtools⁴⁶.

### Comparison Between Species

Gene orthology and phylogeny was assigned using Orthofinder2²⁸. Phylogenetic trees were visualized using iTOL⁴⁷. For TF target gene comparisons, we used a custom python script. We only considered intergenic peaks, and limited the analysis to at most the top 10 target promoters in each organism. We filtered for peaks with a fold-change >=5, p-score >=60, and located <500 bases from the start codon, where p-score is the value assigned by macs2 equal to −log 10 (peak p-value). To avoid matches based on weak binding sites, we filtered out any peaks with a fold-change of less than 5% of the tallest intergenic peak in the same library. We also exclude any TFs that did not perform well, by examining the corresponding peaks in the species from which they originate (E. coli or P. simiae). We defined good performance as having at least one intergenic peak with a fold-change >=15 and p-score >=180. For comparison of target gene similarity between species, we only considered matches in organisms that have at least one putative ortholog of the TF in their own genome. Since in some cases a single organism contributes multiple genes to a single orthogroup, we adjusted for the uniqueness of each target gene by weighting each match based on the number of genes in the corresponding orthogroup. For each target gene set comparison we calculated a p-score by running the same analysis on an equal sized set of randomly selected genes for 10,000 iterations.

### Comparison to Existing Phenotype Data

We downloaded the phenotype dataset for each relevant species from the Fitness Browser web site at http fit.genomic.lbl.gov. We only considered phenotype measurements that were scored as both significant and specific (“specific phenotypes”). From these datasets, we identified cases where the same conditional challenge yielded a specific phenotype assignment for both the TF itself and the TF target gene(s) as predicted by multiDAP.

### Motif Calling and Promoter Analysis

Motifs were called using MEME.⁴⁸ The input sequences used were those flanking the summit position +/−30 bases. For each TF, we only use the top 30 summits (scored by fold-change over background) in the dataset. Significant motifs (E-value <0.05) were manually inspected for quality to exclude motifs that were not found enriched near the center of strong peaks and those that had low total information content. Motifs were mapped against promoter sequences using FIMO⁴⁹ with default options, and only motifs with scores >0 were considered.

We used Tomtom⁵⁰ to compare the E. coli motifs from this study to the motifs published in RegulonDB.²⁶ Motifs were considered to be in agreement if their comparison produced a score with p-value <0.01.

We used 113,676 annotated metagenomic datasets from the Integrated Microbial Genomes (IMG)²⁶ database to extract homologs of E. coli TFs and their corresponding promoter sequences. First, for each E. coli TF, we found the corresponding orthologs in 48 selected bacterial species based on bidirectional best BLAST hits and tabulated each TF orthogroup with conserved Pfam domains found in them. We searched E. coli proteins against predicted genes in metagenomes using MMseqs2″ with E-value 1e-5 and selected all hits which have a start codon (starting with Met) and at least 100 bp upstream sequence from gene. Corresponding promoters were extracted as regions (−100 to +10) around the start codon. Selected orthologs were further filtered to keep only those which have the same Pfam domain(s) and the length within the range of protein lengths of the corresponding orthogroup. To remove the redundant sequences, for each TF, all of its metagenome homologs were clustered using UCLUST⁵² at the percent identity cutoff of 80%, and only one TF and its corresponding promoter were kept for each cluster. Motifs in promoter sequences were predicted using FIMO⁴⁹ with default options.

### LISTING OF REFERENCES CORRESPONDED TO NUMBERED CITATIONS

1. Johnson, D. S., Mortazavi, A., Myers, R. M. & Wold, B. Genome-wide mapping of in vivo protein-DNA interactions. Science 316, 1497-1502 (2007).
2. Barski, A. et al. High-resolution profiling of histone methylations in the human genome. Cell 129, 823-837 (2007).
3. Robertson, G. et al. Genome-wide profiles of STAT1 DNA association using chromatin immunoprecipitation and massively parallel sequencing. Nat. Methods 4, 651-657 (2007).
4. Mikkelsen, T. S. et al. Genome-wide maps of chromatin state in pluripotent and lineage-committed cells. Nature 448, 553-560 (2007).
5. Berger, M. F. & Bulyk, M. L. Protein Binding Microarrays (PBMs) for the Rapid, High-Throughput Characterization of the Sequence Specificities of DNA Binding Proteins. Methods Mol. Biol. Clifton N.J. 338, 245-260 (2006).
6. Oliphant, A. R., Brandl, C. J. & Struhl, K. Defining the sequence specificity of DNA-binding proteins by selecting binding sites from random-sequence oligonucleotides: analysis of yeast GCN4 protein. Mol. Cell. Biol. 9, 2944-2949 (1989).
7. Ellington, A. D. & Szostak, J. W. In vitro selection of RNA molecules that bind specific ligands. Nature 346, 818-822 (1990).
8. Tuerk, C. & Gold, L. Systematic evolution of ligands by exponential enrichment: RNA ligands to bacteriophage T4 DNA polymerase. Science 249, 505-510 (1990).
9. O'Malley, R. C. et al. Cistrome and Epicistrome Features Shape the Regulatory DNA Landscape. Cell 165, 1280-1292 (2016).
10. Bartlett, A. et al. Mapping genome-wide transcription-factor binding sites using DAP-seq. Nat. Protoc. 12, 1659-1672 (2017).
11. Fischer, M. S., Wu, V. W., Lee, J. E., O'Malley, R. C. & Glass, N. L. Regulation of Cell-to-Cell Communication and Cell Wall Integrity by a Network of MAP Kinase Pathways and Transcription Factors in Neurospora crassa. Genetics 209, 489-506 (2018).
12. Mendoza, A. de, Pflueger, J. & Lister, R. Capture of a functionally active methyl-CpG binding domain by an arthropod retrotransposon family. Genome Res. gr.243774.118 (2019) doi:10.1101/gr.243774.118.
13. Galli, M. et al. The DNA binding landscape of the maize AUXIN RESPONSE FACTOR family. Nat. Commun. 9, 4526 (2018).
14. Uygun, S., Azodi, C. B. & Shiu, S.-H. Cis-Regulatory Code for Predicting Plant Cell-Type Transcriptional Response to High Salinity. Plant Physiol. 181, 1739-1751(2019).
15. Brooks, M. D. et al. Network Walking charts transcriptional dynamics of nitrogen signaling by integrating validated and predicted genome-wide interactions. Nat. Commun. 10, 1569 (2019).
16. Ricci, W. A. et al. Widespread long-range cis-regulatory elements in the maize genome. Nat. Plants 5, 1237-1249 (2019).
17. Nitta, K. R. et al. Conservation of transcription factor binding specificities across 600 million years of bilateria evolution. eLife 4, e04837 (2015).
18. Hemberg, M. & Kreiman, G. Conservation of transcription factor binding events predicts gene expression across species. Nucleic Acids Res. 39, 7092-7102 (2011).
19. Kurzchalia, T. V. et al. tRNA-mediated labelling of proteins with biotin. Eur. J. Biochem. 172, 663-668 (1988).
20. Santos-Zavaleta, A. et al. RegulonDB v 10.5: tackling challenges to unify classic and high throughput knowledge of gene regulation in E. coli K-12. Nucleic Acids Res. 47, D212-D220 (2019).
21. Lamers, J., Schippers, B. & Geels, F. Soil-borne diseases of wheat in the Netherlands and results of seed bacterization with Pseudomonas against Gaeumannomyces graminis var. tritici. in Cereal Breeding Related to Integrated Cereal Production 134-139 (Pudoc Wageningen, 1988).
22. Cole, B. J. et al. Genome-wide identification of bacterial plant colonization genes. PLOS Biol. 15, e2002860 (2017).
23. Price, M. N. et al. Mutant phenotypes for thousands of bacterial genes of unknown function. Nature 557, 503-509 (2018).
24. Benson, D. A., Karsch-Mizrachi, I., Lipman, D. J., Ostell, J. & Wheeler, D. L. GenBank. Nucleic Acids Res. 35, D21-D25 (2007).
25. O'Leary, N. A. et al. Reference sequence (RefSeq) database at NCBI: current status, taxonomic expansion, and functional annotation. Nucleic Acids Res. 44, D733-745 (2016).
26. Chen, I.-M. A. et al. IMG/M v.5.0: an integrated data management and comparative analysis system for microbial genomes and microbiomes. Nucleic Acids Res. 47, D666-D677 (2019).
27. Browning, D. F. & Busby, S. J. W. The regulation of bacterial transcription initiation. Nat. Rev. Microbiol. 2, 57-65 (2004).
28. Emms, D. M. & Kelly, S. OrthoFinder: phylogenetic orthology inference for comparative genomics. Genome Biol. 20, 238 (2019).
29. Eraso, J. M. et al. The highly conserved MraZ protein is a transcriptional regulator in Escherichia coli. J. Bacteriol. 196, 2053-2066 (2014).
30. Tamames, J., Gonzalez-Moreno, M., Mingorance, J., Valencia, A. & Vicente, M. Bringing gene order into bacterial shape. Trends Genet. 17, 124-126 (2001).
31. Feng, D.-F., Cho, G. & Doolittle, R. F. Determining divergence times with a protein clock: Update and reevaluation. Proc. Natl. Acad. Sci. 94, 13028-13033 (1997).
32. Grenier, F., Matteau, D., Baby, V. & Rodrigue, S. Complete Genome Sequence of Escherichia coli BW25113. Genome Announc. 2, (2014).
33. Xu, C., Shi, W. & Rosen, B. P. The chromosomal arsR gene of Escherichia coli encodes a trans-acting metalloregulatory protein. J. Biol. Chem. 271, 2427-2432 (1996).
34. Chen, J., Yoshinaga, M., Garbinski, L. D. & Rosen, B. P. Synergistic interaction of glyceraldehydes-3-phosphate dehydrogenase and ArsJ, a novel organoarsenical efflux permease, confers arsenate resistance. Mol. Microbiol. 100, 945-953 (2016).
35. Hall, B. G. & Xu, L. Nucleotide sequence, function, activation, and evolution of the cryptic asc operon of Escherichia coli K12. Mol. Biol. Evol. 9, 688-706 (1992).
36. Ishida, Y., Kori, A. & Ishihama, A. Participation of regulator AscG of the beta-glucoside utilization operon in regulation of the propionate catabolism operon. J. Bacteriol. 191, 6136-6144 (2009).
37. Schiessl, K. T. et al. Phenazine production promotes antibiotic tolerance and metabolic heterogeneity in Pseudomonas aeruginosa biofilms. Nat. Commun. 10, 762 (2019).
38. Swanson, B. L., Colmer, J. A. & Hamood, A. N. The Pseudomonas aeruginosa Exotoxin A Regulatory Gene, ptxS: Evidence for Negative Autoregulation. J. Bacteriol. 181, 4890-4895 (1999).
39. Chen, Y. M., Zhu, Y. & Lin, E. C. The organization of the fuc regulon specifying L-fucose dissimilation in Escherichia coli K12 as determined by gene cloning. Mol. Gen. Genet. MGG 210, 331-337 (1987).
40. Shimada, T., Yokoyama, Y., Anzai, T., Yamamoto, K. & Ishihama, A. Regulatory Role of PlaR (YiaJ) for Plant Utilization in Escherichia coli K-12. Sci. Rep. 9, 20415 (2019).
41. BBMap. SourceForge https://sourceforge.net/projects/bbmap/.
42. Langmead, B. & Salzberg, S. L. Fast gapped-read alignment with Bowtie 2. Nat. Methods 9, 357-359 (2012).
43. Li, H. et al. The Sequence Alignment/Map format and SAMtools. Bioinforma. Oxf. Engl. 25, 2078-2079 (2009).
44. Ramirez, F. et al. deepTools2: a next generation web server for deep-sequencing data analysis. Nucleic Acids Res. 44, W160-W165 (2016).
45. Zhang, Y. et al. Model-based Analysis of ChIP-Seq (MACS). Genome Biol. 9, R137 (2008).
46. Quinlan, A. R. & Hall, I. M. BEDTools: a flexible suite of utilities for comparing genomic features. Bioinformatics 26, 841-842 (2010).
47. Letunic, I. & Bork, P. Interactive Tree Of Life (iTOL) v4: recent updates and new developments. Nucleic Acids Res. 47, W256-W259 (2019).
48. Bailey, T. L. & Elkan, C. Fitting a mixture model by expectation maximization to discover motifs in biopolymers. Proc. Int. Conf Intell. Syst. Mol. Biol. 2, 28-36 (1994).
49. Grant, C. E., Bailey, T. L. & Noble, W. S. FIMO: scanning for occurrences of a given motif. Bioinformatics 27, 1017-1018 (2011).
50. Gupta, S., Stamatoyannopoulos, J. A., Bailey, T. L. & Noble, W. S. Quantifying similarity between motifs. Genome Biol. 8, R24 (2007).
51. Steinegger, M. & Soding, J. MMseqs2 enables sensitive protein sequence searching for the analysis of massive data sets. Nat. Biotechnol. 35, 1026-1028 (2017).
52. Edgar, R. C. Search and clustering orders of magnitude faster than BLAST. Bioinformatics 26, 2460-2461(2010).

It is understood that the examples and embodiments described herein are for illustrative purposes only and that various modifications or changes in light thereof will be suggested to persons skilled in the art and are to be included within the spirit and purview of this application and scope of the appended claims.

All publications, patents, and patent applications cited herein are hereby incorporated by reference with respect to the material for which they are expressly cited.

What is claimed is:

1. A method of identifying transcription factor binding sites in a genomic DNA sample, the method comprising:
transcribing a nucleic acid template encoding a transcription factor of interest in an in vitro transcription reaction to obtain RNA encoding the transcription factor;
translating the RNA in an in vitro translation reaction comprising a tRNA charged with an amino acid labeled with an affinity moiety, wherein the amino acid labeled with the affinity moiety is incorporated into the transcription factor polypeptide in the in vitro translation reaction to generate an affinity-labelled transcription factor;
incubating the affinity-labelled transcription factor with an affinity binding partner immobilized on a solid support to localize the affinity-labelled transcription factors to a discrete region;
incubating the affinity-labeled transcription factor with fragmented genomic DNA obtained from a sample to allow the affinity-labeled transcription factor to bind to transcription factor binding sites sequences present in the fragmented genomic DNA to provide transcription factor-DNA complexes; wherein the fragments of genomic DNA comprise an adaptor oligonucleotide sequence;
washing the solid support to remove unbound fragmented genomic DNA;
processing the fragmented genomic DNA bound to transcription factor immobilized to the solid support for massively parallel sequence analysis;
sequencing the fragment DNA to obtain the sequence of the fragments to which the transcription factor binds.

2. The method of claim 1, wherein the template encoding the transcription factor is generated in an amplification reaction.

3. The method of claim 2, wherein the amplification reaction is PCR.

4. The method of 1, wherein the step of incubating the affinity labeled transcription factor with fragmented genomic DNA is performed before incubation of the affinity labeled transcription factor with the affinity binding partner.

5. The method of claim 1, wherein the amino acid labeled with the affinity moiety comprises a reactive side chain.

6. The method of claim 1, wherein the amino acid labeled with the affinity moiety comprises a hydrophilic or charged side chain.

7. The method of claim 1, the amino acid labeled with the affinity moiety is lysine, arginine, tyrosine, glutamate, aspartate or cysteine.

8. The method of claim 7, wherein the affinity-modified amino acid is lysine.

9. The method of claim 1, wherein the affinity moiety comprises biotin.

10. The method of claim 1, wherein the solid support is a bead.

11. The method of claim 10, wherein the bead is a magnetic bead.

12. The method of claim 1, wherein the processing step comprises an amplification reaction.

13. The method of claim 1, wherein the method is a multiplex reaction comprising genomic DNA from a diversity of samples.

14. A method of evaluating binding interactions of a polypeptide of interest with a nucleic acids, the method comprising:
transcribing a nucleic acid template encoding the polypeptide in an in vitro transcription reaction to obtain RNA encoding the polypeptide;
translating the RNA in an in vitro translation reaction comprising a tRNA charged with an amino acid labeled with an affinity moiety, wherein the amino acid labeled with the affinity moiety is incorporated into the polypeptide in the in vitro translation reaction to generate an affinity-labelled polypeptide;
incubating the affinity-labelled polypeptide with an affinity binding partner immobilized to a solid support to localize the affinity-labelled polypeptide to a discrete region;
incubating the affinity-labelled polypeptide with a population of nucleic acids to evaluate binding of nucleic acids present in the population to the affinity-labelled polypeptides;
washing the solid support to remove unbound candidate polypeptides;
processing the nucleic acids immobilized to the solid support for massively parallel sequence analysis;
sequencing the nucleic acids to obtain the sequence of nucleic acids that bind the affinity-labelled polypeptide.

15. The method of claim 14, wherein the population of nucleic acids are RNA molecules.

16. The method of claim 15, wherein the processing step comprises an RT reaction.

17. The method of claim 14, wherein the population of nucleic acids comprises synthetic oligonucleotide aptamer candidates

18. The method of claim 14, wherein the template encoding the polypeptide of interest is generated in an amplification reaction.

19. The method of claim 18, wherein the amplification reaction is PCR.

20. The method of claim 14, wherein the step of incubating the affinity labeled polypeptide with the population of nucleic acids is performed before incubation of the affinity labeled polypeptide with the affinity binding partner.

21. The method of claim 14, wherein the amino acid labeled with the affinity moiety comprises a reactive side chain.

22. The method of claim 14, wherein the amino acid labeled with the affinity moiety comprises a hydrophilic or charged side chain.

23. The method of claim 14, the amino acid labeled with the affinity moiety is lysine, arginine, tyrosine, glutamate, aspartate or cysteine.

24. The method of claim 23, wherein the affinity-modified amino acid is lysine.

25. The method of claim 14, wherein the affinity moiety comprises biotin.

26. The method of claim 14, wherein the solid support is a bead.

27. The method of claim 26, wherein the bead is a magnetic bead.

28. The method of claim 14, wherein the processing step comprises an amplification reaction.

29. The method of claim 14, wherein the method is a multiplex reaction comprising populations of nucleic acids from a diversity of sources.
